(19) United States
(12) Patent Application Publication
(10) Pub. No.: US 2026/0061182 A1
(43) Pub. Date: Mar. 5, 2026

(54) METHOD OF REDUCING MICROVASCULAR OBSTRUCTIONS BY PROVIDING VENTRICULAR SUPPORT

(71) Applicant: ABIOMED, INC., DANVERS, MA (US)

(72) Inventors: SATHYA DEV UNUDURTHI, Danvers, MA (US); Alfred Kyrollos, Danvers, MA (US); Daniel Berkhoff, Danvers, MA (US)

(73) Assignee: ABIOMED, INC., DANVERS, MA (US)

(21) Appl. No.: 19/310,219

(22) Filed: Aug. 26, 2025

Related U.S. Application Data
(60) Provisional application No. 63/687,591, filed on Aug. 27, 2024.

(51) Int. Cl. A61M 60/32; A61M 60/13; A61M 60/237; A61M 60/422; A61M 60/531

(52) U.S. Cl. CPC A61M 60/32; A61M 60/13; A61M 60/237; A61M 60/422; A61M 60/531

(57) ABSTRACT

The disclosed technology relates to methods of preventing or limiting effects of heart failure in a human patient that has sustained acute myocardial infarction. The method includes determining that the patient is exhibiting acute myocardial infarction, inserting a transvalvular pump at least partially into a heart of the patient, and operating the transvalvular pump to cause blood to flow at a rate of at least 2.5 L/min for at least a support period. The method further includes performing a reperfusion of the patient's heart to restore blood flow to the heart after at least the support period has ended and reducing the amount of tissue actually comprising microvascular obstruction (MVO) of tissue that is at risk of MVO by approximately 20%.

### CROSS REFERENCE TO RELATED APPLICATIONS

This application claims the benefit of priority under 35 U.S.C. § 119 to prior filed U.S. Provisional Ser. No. 63/687,591 , filed Aug. 27, 2024 (Attorney Docket No.: 266489.000010 (ABD041 9USPSP1)), the entire contents of which is hereby incorporated by reference in its entirety as if set forth in full herein.

### TECHNICAL FIELD

Disclosed herein are methods for reducing microvascular obstruction (MVO) following acute myocardial infarction (AMI) and subsequent successful reperfusion.

### BACKGROUND

Mechanical circulatory support devices, such as intracardiac or intravascular blood pumps (sometimes referred to as intravalvular blood pumps) may be introduced in the heart to deliver blood from the heart into an artery. Such devices are often introduced to support the function of the heart after a patient suffers a cardiac episode. One such class of devices is the Impella® line of blood pumps. Some blood pump assemblies may be introduced percutaneously through the vascular system during a cardiac procedure. Specifically, blood pump assemblies can be inserted via a catheterization procedure through the femoral artery or the axillary/subclavian artery, into the ascending aorta, across the aortic valve and into the left ventricle. The inserted blood pump assembly may be configured to pull blood from the left ventricle of the heart through a cannula and expels the blood into the aorta. A blood pump assembly may also be configured to pull blood from the inferior vena cava and to expel blood into the pulmonary artery. Some mechanical circulatory support devices are powered by an on-board motor, while others are powered by an external motor and a drive cable.

### BRIEF SUMMARY

The disclosed technology relates to methods of preventing or limiting effects of heart failure in a human patient that has sustained acute myocardial infarction. The method includes determining that the patient is exhibiting acute myocardial infarction, inserting a transvalvular pump at least partially into a heart of the patient, and operating the transvalvular pump to cause blood to flow at a rate of at least 2.5 L/min for at least a support period. The method further includes performing a reperfusion of the patient's heart to restore blood flow to the heart after at least the support period has ended and reducing the amount of tissue actually comprising microvascular obstruction (MVO) of tissue that is at risk of MVO by approximately 20%.

The disclosed technology further includes a method comprising inserting a transvalvular pump at least partially into a heart of the patient and operating the transvalvular pump to cause blood to flow at a rate of at least 2.5 L/min for at least a support period. The method further includes performing, after the support period, a reperfusion of the patient's heart to restore blood flow to the heart and reducing a percentage of tissue comprising infarction due to microvascular obstruction (MVO) of tissue at risk of comprising infarction by approximately 9%.

The disclosed technology further includes a method comprising, in response to determining that the patient is exhibiting acute myocardial infarction, inserting a transvalvular pump at least partially into a heart of the patient and operating the transvalvular pump to cause blood to flow at a rate of at least 2.5 L/min for at least a support period. The method further includes performing a reperfusion of the patient's heart to restore blood flow to the heart and reducing the amount of detectable neutrophils in tissue of the heart by more than half.

### BRIEF DESCRIPTION OF DRAWINGS

The patent or application file contains at least one drawing executed in color. Copies of this patent or patent application publication with color drawing(s) will be provided by the Office upon request and payment of the necessary fee.

While the specification concludes with claims, which particularly point out and distinctly claim the subject matter described herein, it is believed the subject matter will be better understood from the following description of certain examples taken in conjunction with the accompanying drawings, in which like reference numerals identify the same elements. The figures depict one or more implementations of the inventive devices, by way of example only, not by way of limitation.

FIG. 1A is an illustration of an acute myocardial infarction.

FIG. 1B is an illustration of a stent implantation restoring coronary blood flow.

FIG. 1C is an illustration of an example of no reflow phenomenon.

FIG. 2 is a magnetic resonance image of microvasculature showing microvascular obstruction (MVO) and resultant infarction.

FIG. 3 is an illustration of various molecular mechanisms that cause MVO.

FIG. 4 is a chart illustrating a correlation of mortality rate and heart failure hospitalization over time to the extent of microvascular obstruction in patients.

FIG. 5 shows an illustrative cardioprotective system, according to an example of the disclosed technology.

FIG. 6 shows an illustrative device and method of supporting a patient's heart that has sustained myocardial infarction, according to an example of the disclosed technology.

FIG. 7 is an illustration of an example intravalvular pump inserted into a heart of a patient, according to an example of the disclosed technology.

FIG. 8 is a flow chart of a procedure conducted on swine of a control group in a pilot study.

FIG. 9 is a flow chart of a procedure conducted on swine of a study group (unloaded heart) in a pilot study, according to an example of the disclosed technology.

FIG. 10 illustrates an example of sections of a heart that is cut to study the effects of MVO, according to an example of the disclosed technology.

FIGS. 11A and 11B illustrate sections of a heart stained with S-thioflavin to study the presence of MVO, according to an example of the disclosed technology.

FIG. 12 illustrates sections of a heart of a swine in the control group that is cut and stained with S-thioflavin to study the presence of MVO.

FIG. 13 illustrates sections of a heart of a swine in the study group (unloaded heart) that is cut and stained with S-thioflavin to study the presence of MVO, according to an example of the disclosed technology.

FIG. 14 is a table illustrating results of the pilot study, according to an example of the disclosed technology.

FIG. 15A shows images of tissue of a heart of a swine in the control group exhibiting extensive interstitial edema.

FIG. 15B shows images of tissue of a heart of a swine in the study group (unloaded) exhibiting no noticeable interstitial edema, according to an example of the disclosed technology.

FIG. 16A shows images of tissue of a heart of a swine in the control group exhibiting extensive pericapillary edema and cellular dipedesis.

FIG. 16B shows images of tissue of a heart of a swine in the study group (unloaded) exhibiting reduced peri capillary edema and cellular dipedesis, according to an example of the disclosed technology.

FIG. 17A shows images of tissue of a heart of a swine in the control group exhibiting neutrophil infiltration.

FIG. 17B shows images of tissue of a heart of a swine in the study group (unloaded) exhibiting reduced neutrophil infiltration, according to an example of the disclosed technology.

FIG. 18 shows a graph of quantitative estimation of neutrophil infiltration in infarct tissue of the control group, the study group (unloaded), and remote tissue, respectively, according to an example of the disclosed technology.

FIG. 19A shows images of tissue of a heart of a swine in the control group exhibiting reduced cardiomyocyte integrity.

FIG. 19B shows images of tissue of a heart of a swine in the study group (unloaded) exhibiting preserved cardiomyocyte integrity, according to an example of the disclosed technology.

FIG. 20A shows images of tissue of a heart of a swine in the control group exhibiting reduced intercalated disk integrity in cardiomyocytes.

FIG. 20B shows images of tissue of a heart of a swine in the study group (unloaded) exhibiting preserved intercalated disk integrity in cardiomyocytes, according to an example of the disclosed technology.

FIG. 21 illustrates components of cardiomyocytes for illustrative purposes.

FIG. 22 illustrates a flow chart of a method of treating acute myocardial infarction in a patient, according to an example of the disclosed technology.

### DETAILED DESCRIPTION

Aspects of the present disclosure are described in detail with reference to the drawing figures wherein like reference numerals identify similar or identical elements. It is to be understood that the disclosed aspects are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items. In the claims, as well as in the specification above, all transitional phrases such as “comprising,” “including”, “carrying”, “having”, “containing”, “involving”, “holding”, “composed of”, and the like are to be understood to be open-ended, i.e., to mean including but not limited to. Only the transitional phrases “consisting of” and “consisting essentially of” shall be closed or semi-closed transitional phrases, respectively.

As used herein, the terms “proximal” and “distal” refer to positions relative to a physician or operator of the intravalvular blood pump. Thus, “proximal” indicates a position that is closer to the physician or operator or a direction that points towards the physician or operator, and “distal” indicates a position that is farther from the physician or operator or a direction that points away from the physician or operator.

As used herein, “operator” can include a doctor, surgeon, technician, or any other individual or instrumentation associated with delivery and operation of a mechanical circulatory support device in a human patient.

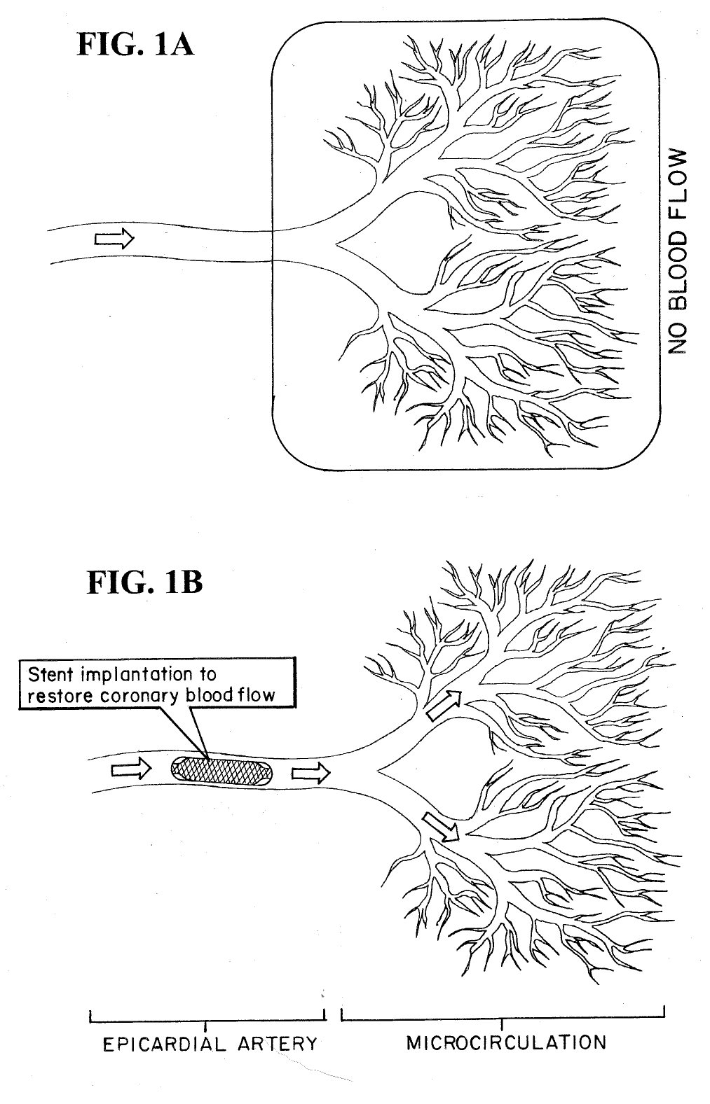

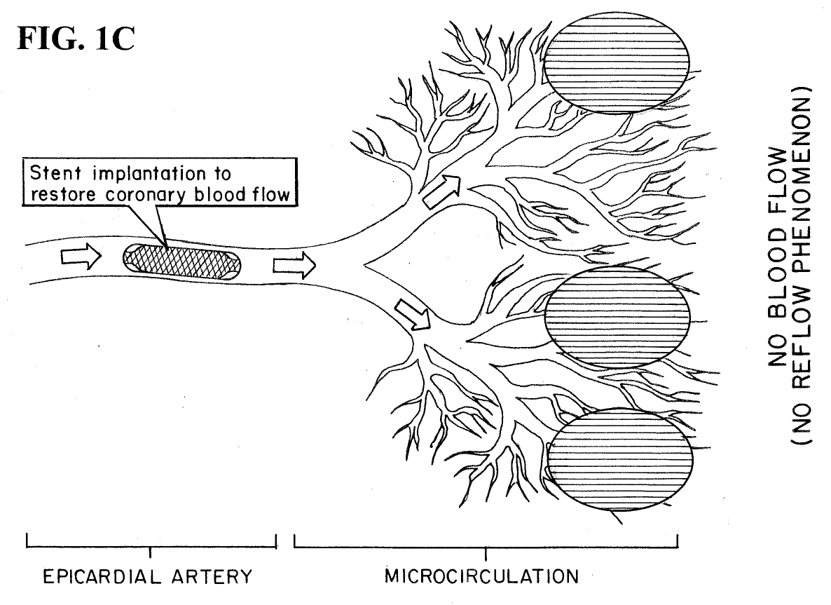

Acute myocardial infarction (AMI) due to occlusion of a coronary artery (as illustrated in FIG. 1A) is a major cause of global morbidity and mortality in humans. The current paradigm for AMI therapy focuses on primary reperfusion, which rapidly restores coronary artery blood flow (as illustrated in FIG. 1B) as soon as possible after AMI, to re-establish myocardial oxygen supply. Despite timely reperfusion, however, up to 25% of patients experiencing their first AMI will develop heart failure (HF) within a year. Contemporary ST-segment elevation AMI (STEMI) in-hospital management focuses on reducing door to balloon (DTB) time to reduce infarct size. However, despite intense resource allocation to achieve DTB times under 90 minutes, the incidence of post-AMI heart failure remains high. For every 5% increase in myocardial infarct size, 1-year all-cause mortality and HF hospitalizations increase by 20%, which imposes a significant burden on healthcare resources. For these reasons, new approaches to limit myocardial damage and subsequent ischemic HF remain a significant unmet need for AMI patients.

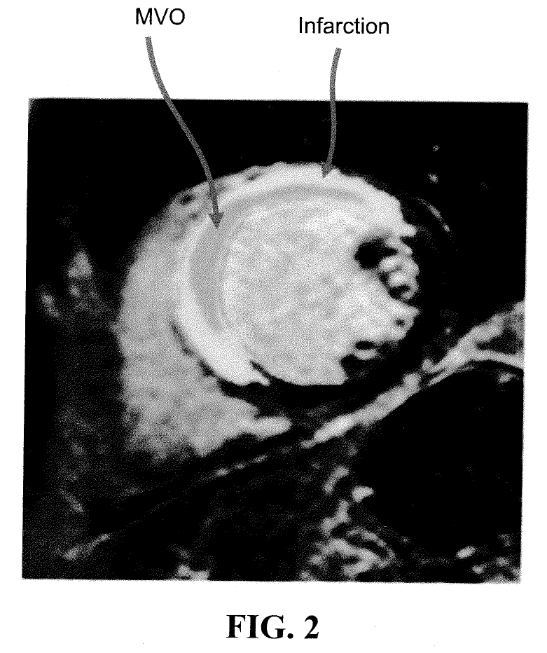

One explanation for these poor outcomes is that primary reperfusion paradoxically may worsen myocardial damage, known as ischemia-reperfusion injury (IRI). One result of IRI includes a condition sometimes referred to as the “no reflow phenomenon.” As illustrated in FIG. 1C, no reflow phenomenon is the failure of blood to reperfuse in ischemic areas after the physical obstruction has been removed or bypassed. Microvascular obstruction (MVO) is the underlying cause of no reflow phenomenon and is common in patients with heart failure. FIG. 2 is a magnetic resonance image of microvasculature showing MVO and resultant infarction. MVO is shown in orange while infarction is shown in yellow.

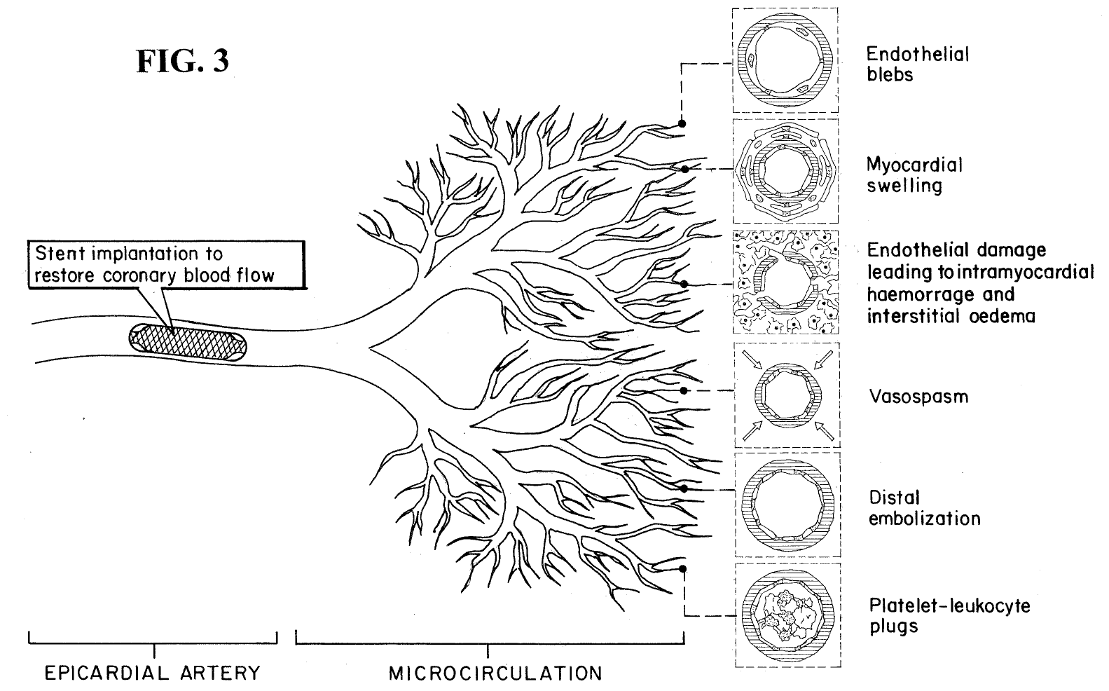

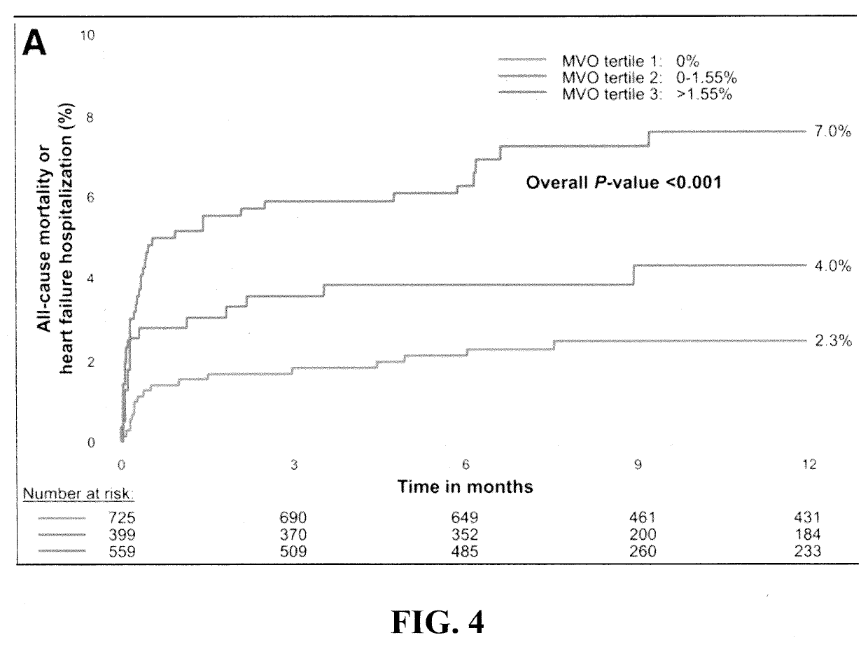

Molecular mechanisms underlying MVO include endothelial blebs, myocardial swelling, endothelial damage leading to intramyocardial hemorrhage and interstitial oedema, vasospasm, distal embolism, and platelet-leukocyte plugs. These various underlying molecular mechanisms are illustrated further in FIG. 3. MVO causes myocardial tissue hypoperfusion even despite successful reperfusion at the epicardial level. As shown in FIG. 4, as the extent of MVO in patients increases, the mortality rate and patient hospitalizations due to heart failure also increase. For example, for every 1% increase in MVO, the hazard of ‘all cause mortality’ or ‘heart failure hospitalization’ increases by 11%.

Prior attempts to limit IRI and MVO include vascular conditioning approaches to activate reperfusion injury salvage kinase (RISK) pathway activity and pharmacologic approaches, but the clinical benefit of those approaches has not necessarily been optimal. A critical barrier to these cardioprotective strategies is the requirement for rapid coronary reperfusion—they potentially leave insufficient time for any therapeutic impact on myocardial injury. Thus, there exists a need for improved strategies to limit myocardial damage by promoting cardioprotective mechanisms that reduce or eliminate IRI, including MVO. Further details about AMI and the technology disclosed herein are included in the Appendix filed herewith, all of which is incorporated by reference as if fully set forth herein.

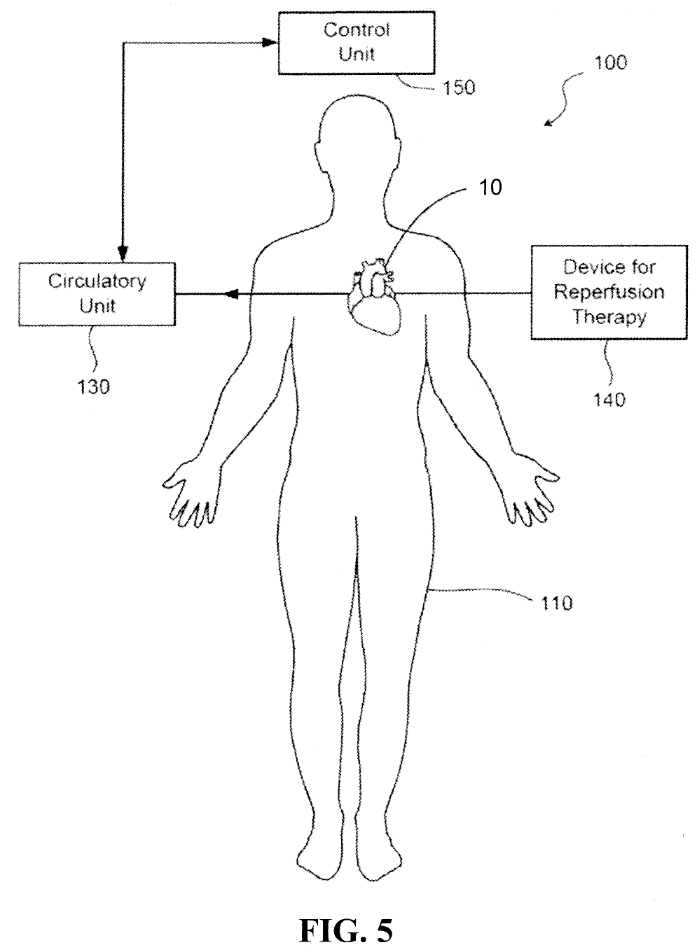

FIG. 5 illustrates a system 100 for providing a combination of mechanical support and Primary Reperfusion according to an implementation of the present disclosure. System 100 aims to limit myocardial damage in a human patient 110 who has experienced AMI in the heart 10. The system 100 comprises a circulatory unit 130 and a device (or other source) for providing reperfusion therapy 140. The circulatory unit 130 is in communication with a control unit 150. Control unit 150 may monitor signals issued by the circulatory unit 130 and, accordingly, control the operation of the devices (or other source) comprising the circulatory unit 130. These signals may be indicative of any one of the following: the operational state of the circulatory unit 130, the position and state of the device for reperfusion therapy 140, and the state of the patient's heart. Samples from the AMI patient, e.g. blood or cardiac tissue, may be obtained from either the circulatory unit 130 or the device for reperfusion therapy 140, or from a biopsy or other source, for characterization and further testing. This may be done via a testing kit or a laboratory to extract various indicia from these samples so that they can be monitored by a clinician. Such indicia may include, for example, the myocardial infarction scar size, and associated parameters that will be detailed in the following sections.

The circulatory unit 130 comprises a mechanical circulatory support device that can be inserted, for example, in the left ventricle of the patient's heart. Such a mechanical circulatory support device is capable of changing the blood flow above and beyond the actual cardiac output of the heart 10. For example, the mechanical circulatory support device may be inserted into the left ventricle of the heart of a patient with AMI and actuated to unload the heart by pumping blood out of the ventricle. This can assist the heart in several possible ways. For example, the myocardium wall stress is reduced. This is beneficial as the mechanism of unloading may assist in myocardial salvage and repair. According to an implementation of the present disclosure, the mechanical circulatory support device may comprise a transvalvular microaxial blood pump. Examples of such blood pumps include, but are not limited to, Impella 2.5™ and Impella CP® by Abiomed, Inc., Danvers, MA. Other types of mechanical circulatory support devices may be used to assist the heart, such as extracorporeal pumps. For example, extracorporeal membrane oxygenation (ECMO) or intraaortic balloon pumps may be used. In some adaptations a transvalvular pump is used in combination with another such device.

In addition to the mechanical circulatory support device, the circulatory unit 130 may also comprise additional pump devices that assist with the unloading of the heart. Examples of such pump assist devices include, but are not limited to, any one of the following: an intra-aortic balloon pump, and an extracorporeal membrane oxygenation (ECMO) pump. For example, a transvalvular pump may unload the heart while a balloon pump or ECMO device is applied to further assist the patient. Additionally, the circulatory device may comprise a cannula portion in fluid communication with a pump in which the distal end of the cannula may be positioned within the heart of the patient, and the pump may be positioned at any one of: (a) within the heart with the cannula, (b) outside the heart but within the patient, and (c) outside the patient.

In an implementation of the present disclosure, the device 140 is used to administer reperfusion therapy to the patient undergoing AMI. Such reperfusion therapy includes, for example, primary percutaneous coronary intervention (PCI). These procedures may involve the use of a coronary stent delivered into the distal left anterior descending artery (LAD). In certain embodiments, reperfusion therapy 140 may comprise drug or medicament that is capable of assisting in fibrinolysis, thereby providing reperfusion therapy either in combination with or as an alternative to a stent or other device.

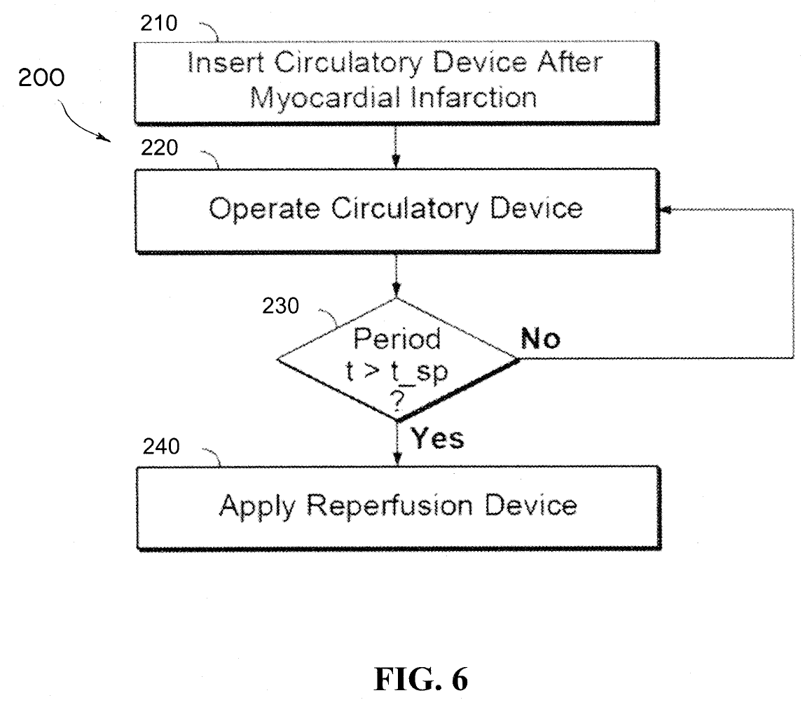

FIG. 6 shows a flowchart of an illustrative method 200 for unloading the left ventricle of the heart in a patient with AMI. The method 200 starts with inserting 210 a circulatory device, such as the mechanical circulatory device of the circulatory unit 130 in FIG. 5, into the patient after myocardial infarction. Such insertion may be achieved by using a vascular access sheath deployed into the right internal jugular vein, left carotid artery, and one or more femoral arteries and veins of the patient.

The method 200 further includes operating 220 the circulatory device to support the heart, for example by unloading the patient's heart after myocardial infarction. Here the circulatory device is operated to achieve a pumping rate of at least 2.5 L/min of blood flow from the left ventricle of the heart. In certain implementations, the circulatory device is operated to achieve a blood flow rate from the left ventricle of the heart of at least 3.5 L/min of blood flow per cardiac output. As will be appreciated, the circulatory device may be operated to achieve any suitable pumping rate of blood flow from the left ventricle of the heart. The unloading is performed for a period 230 (the support period t_sp) that is sufficiently long so as to facilitate a reduction in infarct size. In some implementations, operation of the circulatory device is terminated after the support period t_sp has elapsed. In other implementations, the support period is merely used as a marker to indicate the elapse of time t_sp since the circulatory device has commenced operation, and operation of the circulatory device need not be stopped after t_sp has elapsed. According to some implementations, the support period t_sp is greater than 15 mins. In other implementations, the support period t_sp is greater than 15 minutes and less than 30 mins, greater than 30 minutes and less than 45 minutes, greater than 30 minutes and less than 60 minutes, greater than 45 minutes and less than 60 minutes, or the support period t_sp can be greater than 60 minutes.

After the heart has been unloaded in step 230 for the support period, the method 200 includes applying 240 a reperfusion therapy to the patient's heart. Reperfusion therapy is administered using a reperfusion device. According to an implementation of the present disclosure, reperfusion therapy may be applied to the patient's heart after unloading the left ventricle of the heart. In other implementations, reperfusion therapy may be applied to a patient's heart while the left ventricle is still being unloaded by the circulator unit. In this implementation, the parallel use of the reperfusion device and the circulatory device is only carried out after the heart is unloaded with the circulatory device for the length of the support period t_sp.

Supporting the heart after MI with mechanical circulatory support prior to applying reperfusion therapy has a beneficial effect on the patient's heart as will be further illustrated below. One or more benefits may be detected in tissue or blood samples taken from the patient, conducting an MRI, conducting an autopsy, etc. Such benefits may include, but are not limited to, one or more of the following results: reducing MVO; reducing neutrophil infiltration; reducing perivascular and interstitial edema; and reducing cardiomyocyte damage. These results can be achieved using the systems and methods identified in the present disclosure.

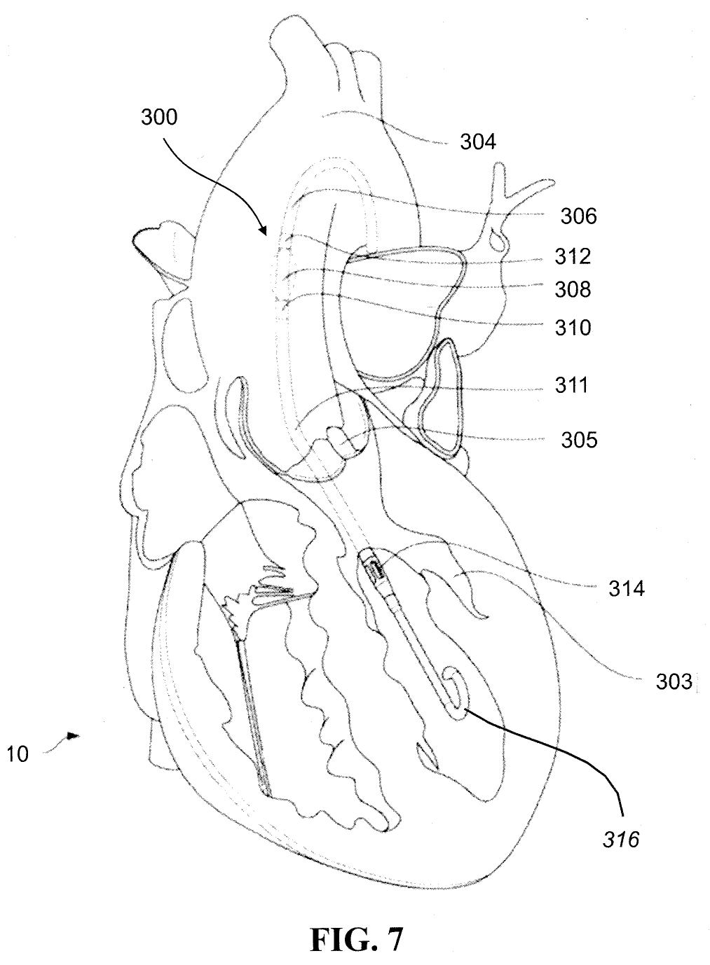

FIG. 7 shows an exemplary transvalvular pump 300 located in a heart 10. The heart 10 includes a left ventricle 303, aorta 304, and aortic valve 305. The transvalvular heart pump 300 includes a catheter 306, a motor 308, a pump outlet 310, a cannula 311, a pump inlet 314, and a pressure sensor 312. The motor 308 is coupled at its proximal end to the catheter 306 and at its distal end to the cannula 311. The motor 308 also drives a rotor (not visible in figure) which rotates to pump blood from the pump inlet 314 through the cannula 311 to the pump outlet 310. The cannula 311 is positioned across the aortic valve 305 such that the pump inlet 314 is located within the left ventricle 303 and the pump outlet 310 is located within the aorta 304. The transvalvular heart pump 300 may also include an atraumatic distal tip 316. In some embodiments, the transvalvular heart pump 300 may not include an traumatic distal tip. In some embodiments, the cannula 311 may be expandable. In such embodiments, there may be a cage surrounding the rotor. In some aspects, both the rotor and the cage may be made of material(s) and/or have a structure configured such that the rotor and the cage are configured to be compressible. This configuration allows the transvalvular heart pump 300 to pump blood from the left ventricle 303 into the aorta 304 to support cardiac output and thereby unload the heart 10. As will be explained further herein, unloading the heart 10 can provide many health benefits to patients experiencing acute myocardial infarction.

The transvalvular heart pump 300 pumps blood from the left ventricle into the aorta in parallel with the native cardiac output of the heart 10. The blood flow through a healthy heart averages about 5 liters/minute, and the blood flow through the transvalvular heart pump 300 can be a similar or different flow rate. For example, the flow rate through the transvalvular heart pump 300 can be 0.5 liters/minute, 1 liter/minute, 1.5 liters per minute, 2 liters/minute, 2.5 liters/minute, 3 liters/minute, 3.5 liters/minute, 4 liters/minute, 4.5 liters/minute, 5 liters/minute, greater than 5 liters/minute or any other suitable flow rate.

The motor 308 of the transvalvular heart pump 300 can vary in any number of ways. For example, the motor 308 can be an electric motor. The motor 308 can be operated at a constant rotational velocity to pump blood from the left ventricle 303 to the aorta 304. Operating the motor 308 to maintain a constant rotor speed generally requires supplying the motor 308 with varying amounts of current because the load on the motor 308 varies during the different stages of the cardiac cycle of the heart 10. For example, when the mass flow rate of blood into the aorta 304 increases (e.g., during systole), the current required to operate the motor 308 increases. This change in motor current can thus be used to help characterize cardiac function. An electric motor current may be measured, or alternatively a magnetic field current may be measured. Detection of mass flow rate using motor current may be facilitated by the position of the motor 308, which is aligned with the natural direction of blood flow from the left ventricle 303 into the aorta 304. Detection of mass flow rate using motor current may also be facilitated by the small size and/or low torque of the motor 308. The motor 308 of FIG. 7 has a diameter of about 4 mm, but any suitable motor diameter may be used provided that the rotor-motor mass is small enough to be influenced by the inertia of pulsatile blood. The rotor-motor mass may be influenced by the pulsatile mass flow of blood to produce a discernable and characterizable effect on the motor parameter. In some implementations, the diameter of the motor 308 is less than 4 mm.

In certain implementations, one or more motor parameters other than current, such as power delivered to the motor 308, speed of the motor 308, or electro-magnetic field are measured. In some implementations, the motor 308 in FIG. 7 operates at a constant velocity. In some implementations, the motor 308 may be external to the patient and may drive the rotor by an elongate mechanical transmission element, such as a flexible drive shaft, drive cable, or a fluidic coupling.

The pressure sensor 312 of the transvalvular heart pump 300 can be an integrated component (as opposed to separate diagnostic catheter) and can be configured to detect pressure at various locations of the system 300 such as adjacent to a proximal end of the motor 308. In certain implementations, the pressure sensor 312 of the transvalvular heart pump 300 can be disposed on the cannula 311, on the catheter 306, on a portion of the system 300 external to the patient's body, or in any other suitable location. The pressure sensor 312 can detect blood pressure in the aorta 304 when the transvalvular heart pump 300 is properly positioned in the heart 10, or for right heart support devices can detect pressure in the inferior vena cava (IVC) or the pulmonary artery. The blood pressure information can be used to properly place the transvalvular heart pump 300 in the heart 10. For example, the pressure sensor 312 can be used to detect whether the pump outlet has passed through the aortic valve 305 into the left ventricle 303 which would only circulate blood within the left ventricle 303 rather than transport blood from the left ventricle 303 to the aorta 304. The pressure sensor in FIG. 7 detects the absolute pressure at a certain point in the patient's vasculature, for example, in the aorta. In other examples, the pressure sensor detects absolute pressure in the pulmonary artery or venous system. In other examples, the pressure sensor detects the pressure head or the delta pressure in the system, which can be equal to the aortic pressure less the left-ventricular pressure.

The transvalvular heart pump 300 can be inserted in various ways, such as by percutaneous insertion into the heart 10. For example, the transvalvular heart pump 300 can be inserted through a femoral artery (not shown), through an axillary artery (not shown), through the aorta 304, across the aortic valve 305, and into the left ventricle 303. In certain implementations, the transvalvular heart pump 300 is surgically inserted into the heart 10. In some implementations, the transvalvular heart pump 300, or a similar system adapted for the right heart, is inserted into the right heart. For example, a right heart pump similar to the transvalvular heart pump 300 can be inserted through the inferior vena cava, bypassing the right atrium and right ventricle, and extending into the pulmonary artery. In certain implementations, the transvalvular heart pump 300 may be positioned for operation in the vascular system outside of the heart 10 (e.g., in the aorta 304). By residing minimally invasively within the vascular system, the transvalvular heart pump 300 is sufficiently sensitive to allow characterization of native cardiac function.

### Pilot Study—Mechanical Unloading of Left Ventricle Before Reperfusion

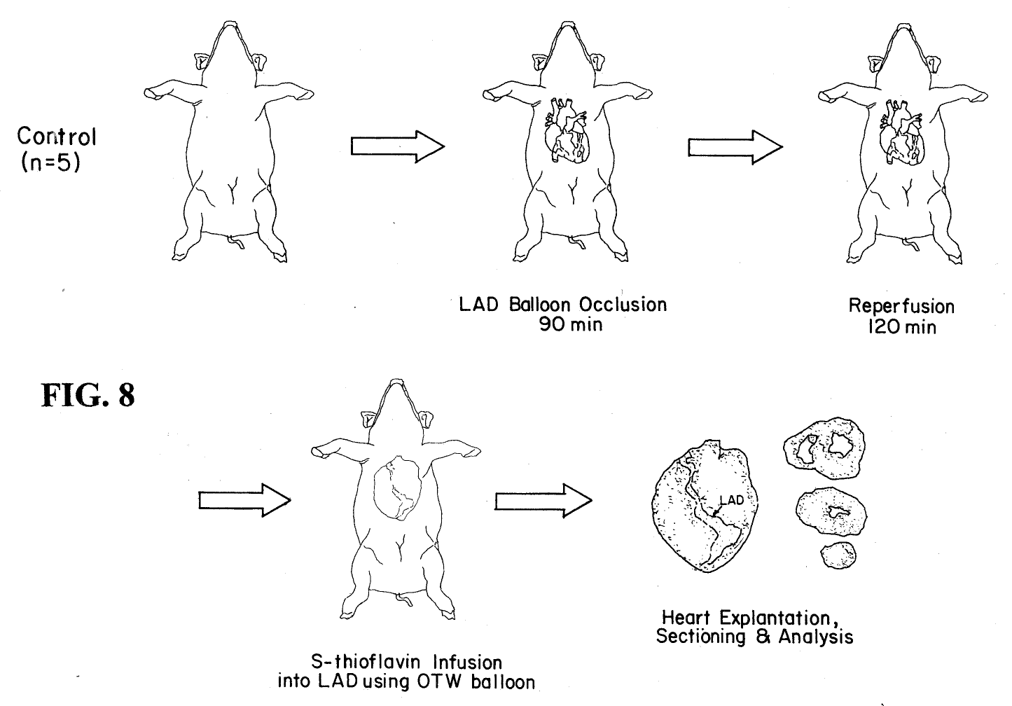

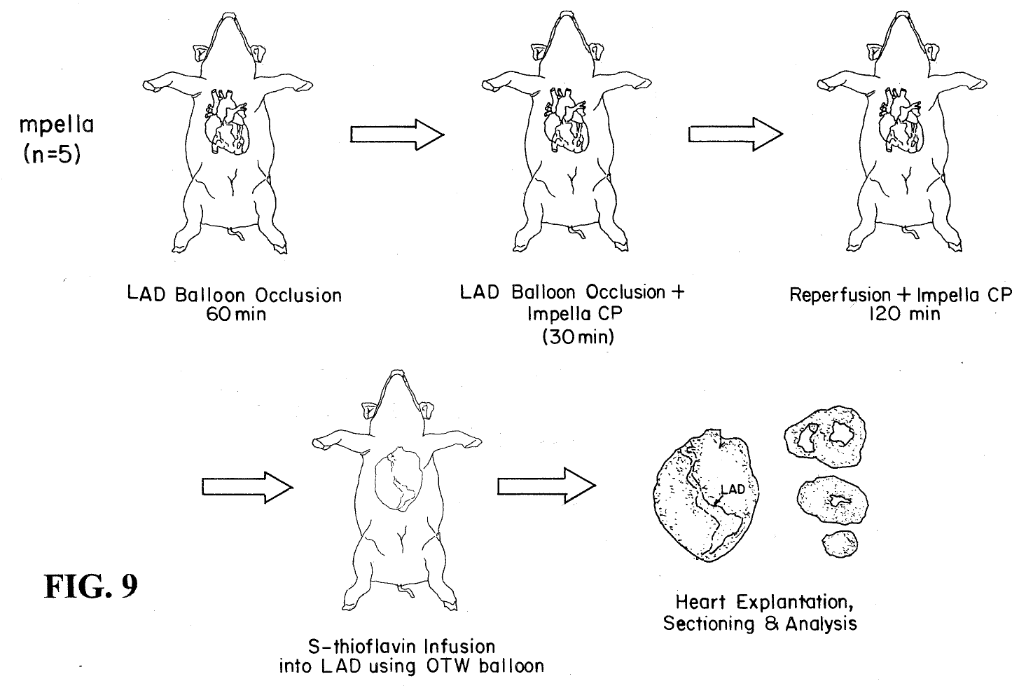

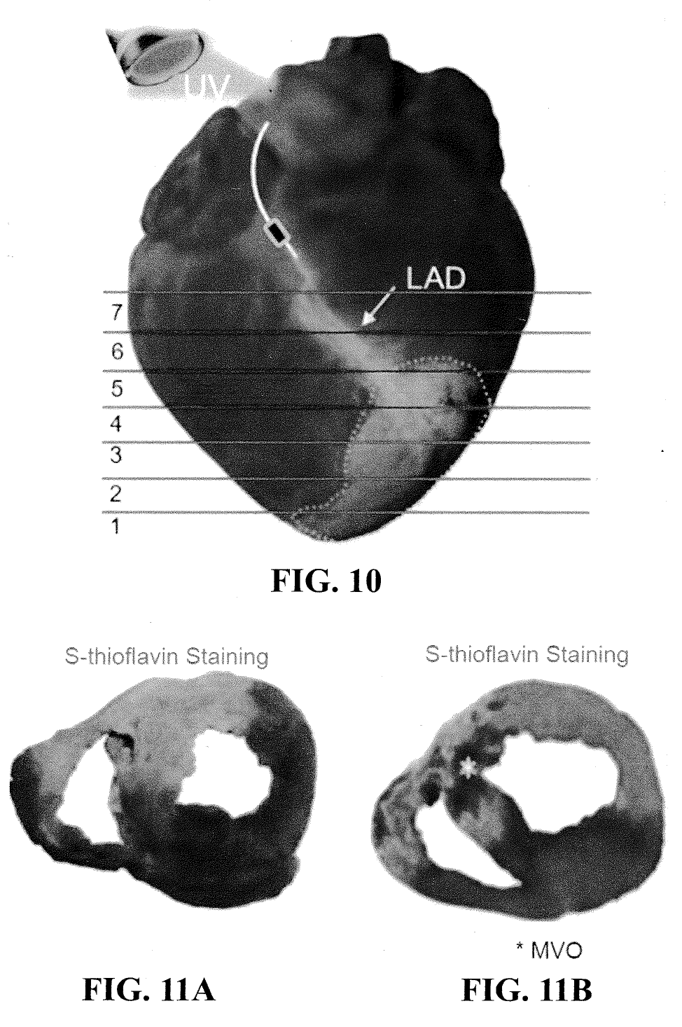

The benefits of operating a transvalvular pump were studied to determine the effects of unloading the heart following acute myocardial infarction. As further illustrated in FIGS. 8 and 9, eleven swine (43-52 kg) underwent a 90-minute LAD balloon occlusion-induced anterior STEMI followed by 120 minutes of reperfusion. The control group (n=6) underwent 90-min of unassisted ballon-occlusion induced STEMI followed by 120 minutes of spontaneous reperfusion (FIG. 8). In contrast, after the first 60 minutes of occlusion, LV unloading was started for the swine in the unloading group (n=5) (FIG. 9) and was continued throughout the entire 120-minute reperfusion period. Before euthanasia, 4% thioflavin-S (TS) was injected into the left anterior descending artery. The left ventricle was then sectioned horizontally into 10-mm thick myocardial rings (as shown in FIG. 10) and photographed under UV light (as shown in FIGS. 11A and 11B) to determine areas at risk (TS positive), MVO (lack of TS staining within the TS positive area), and remote zones (contiguous areas unstained by TS). Afterward, the rings were submerged in 2,3,5-triphenyl tetrazolium chloride (TTC) for 20 minutes and photographed under ambient light. Myocardium samples were collected for histology and immunohistochemistry.

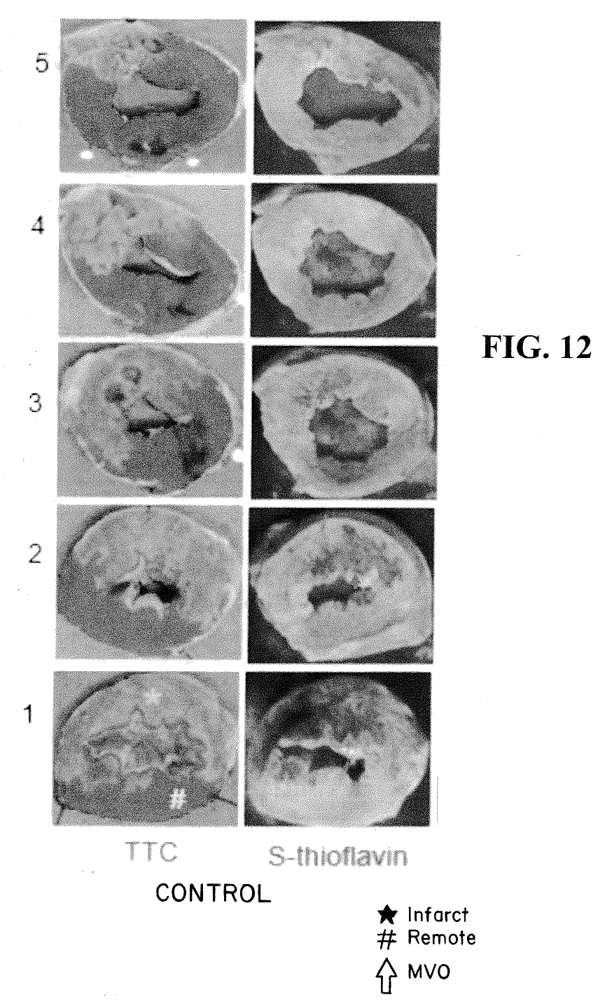

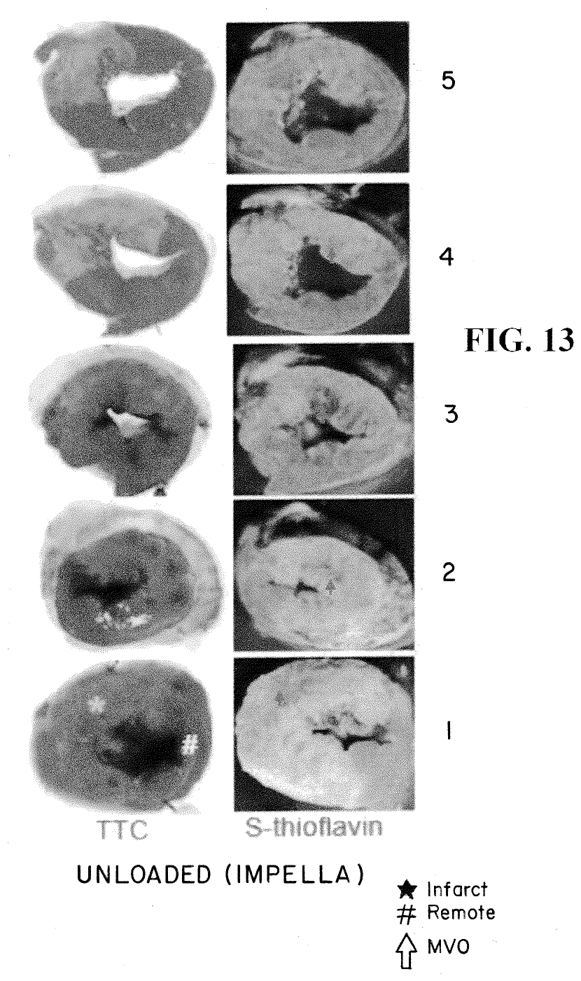

FIG. 12 illustrates various sections of a heart of a swine in the control group while FIG. 13 illustrates various sections of a heart of a swine in the Impella group (unloaded heart). In FIGS. 12 and 13, areas of tissue having infarction are shown with “*”, areas of tissue that were remote from the blockage as shown with a “#”, and areas of the tissue having MVO are shown with an arrow. As shown, the heart of the swine in the control group as well as the heart of the swine in the Impella group both had some areas with infarction and some areas that were remote, however, the heart of the swine in the control group had significantly more MVO than the heart of the swine in the Impella group. These results show that unloading the heart for a support period t_sp before reperfusion reduces the amount of MVO thereby leaving the heart in better condition to recover and reducing the likelihood of heart failure.

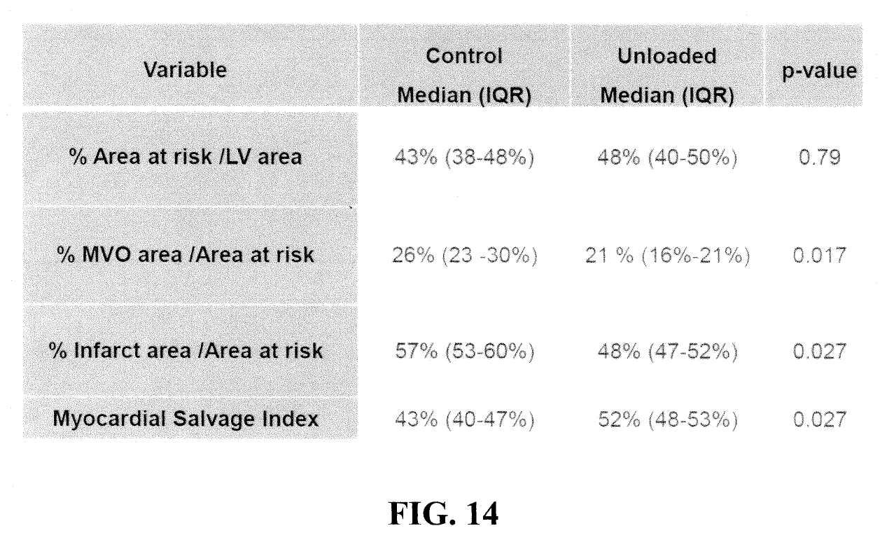

After analyzing the tissue of the heart of each swine, the results were collected and statistically analyzed. As shown in FIG. 14, the median percentage of the area at risk relative to the LV area was similar between the Impella group and the control group (48% [IQR 40-50%] vs. 43% [IQR 38-48%], respectively; p =0.79), yet significant reductions were observed in the Impella group, compared to controls, for MVO areas relative to the area at risk (21% [IQR 16-21%] vs. 26% [IQR 23-30%], p=0.017). That is, of the tissue at risk for MVO, the swine in the Impella group exhibited 5% less tissue actually comprising MVO compared to the control group. Stated otherwise, by unloading the heart for the support period t_sp, the amount of tissue exhibiting MVO of the tissue at risk of MVO was reduced by 20%, thereby reducing the likelihood of heart failure for the swine in the Impella group.

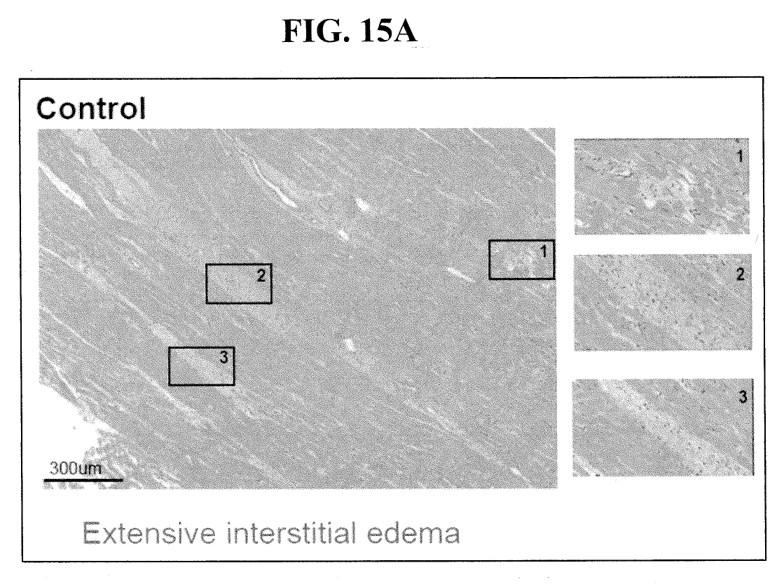

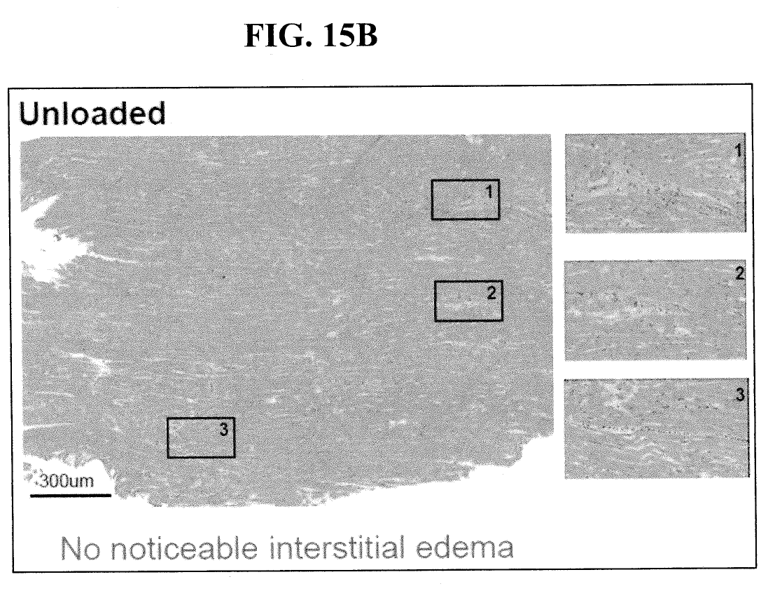

As further shown in FIG. 14, the infarct area relative to the area at risk as also reduced for the Impella group (48% [IQR 47-52%] vs. 57% [IQR 53-60%], p=0.027). That is, the Impella group exhibited 9% less infarction due to MVO in tissue at risk of MVO than the control group. Stated otherwise, by unloading the heart for the support period t_sp, the amount of tissue exhibiting infarction was greatly reduced, thereby reducing the likelihood of heart failure for the swine in the Impella group. Similarly, the myocardial salvage index was significantly higher in the Impella group compared to controls (52% [IQR 48-53%] vs. 43% [IQR 40-47%], p =0.027). In addition, Impella CP-assisted reperfusion reduced neutrophil infiltration, interstitial edema, and cardiomyocyte damage (FIGS. 15A-20B). These results illustrate the significant advantages of unloading the heart with an intravalvular pump for a support period t_sp before reperfusion.

FIG. 15A shows images of tissue of a heart of a swine in the control group while FIG. 15B shows images of tissue of a heart of a swine in the Impella group (unloaded). As shown, the tissue of the heart of the swine in the control group exhibited extensive interstitial edema while the tissue of the heart of the swine in the Impella group exhibited no noticeable interstitial edema. That is, unloading the heart with an intravalvular pump for a support period t_sp before reperfusion was shown to reduce or even eliminate interstitial edema in the tissue.

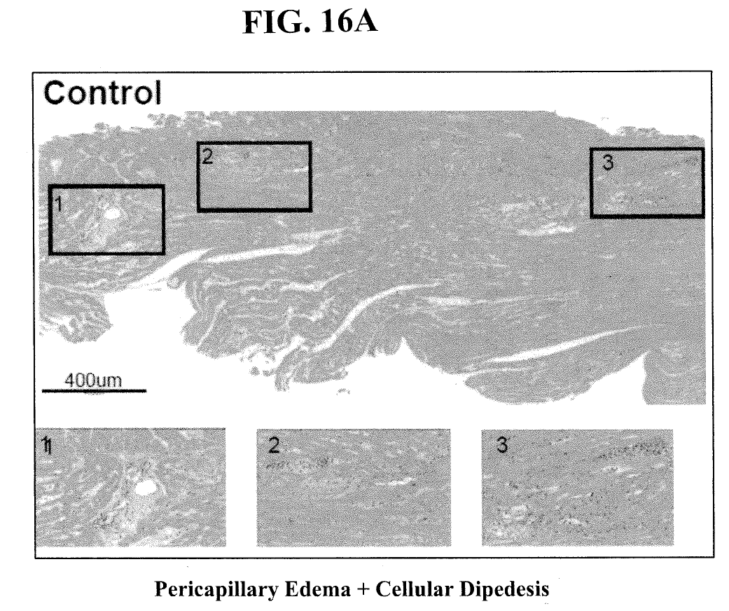

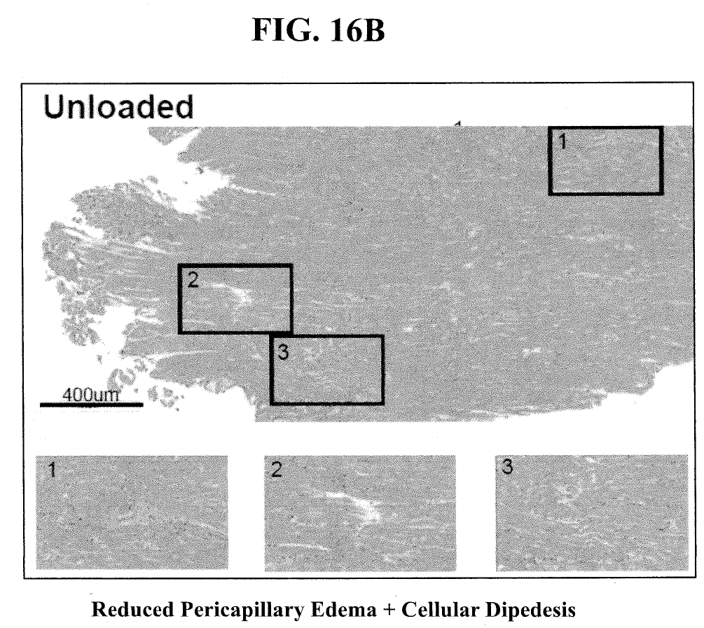

FIG. 16A shows images of tissue of a heart of a swine in the control group while FIG. 16B shows images of tissue of a heart of a swine in the Impella group (unloaded). As shown, the tissue of the heart of the swine in the control group exhibited extensive pericapillary edema and cellular dipedesis while the tissue of the heart of the swine in the Impella group exhibited reduced pericapillary edema and cellular dipedesis. That is, unloading the heart with an intravalvular pump for a support period t_sp before reperfusion was shown to reduce pericapillary edema and cellular dipedesis which further supports the health benefits of the disclosed technology.

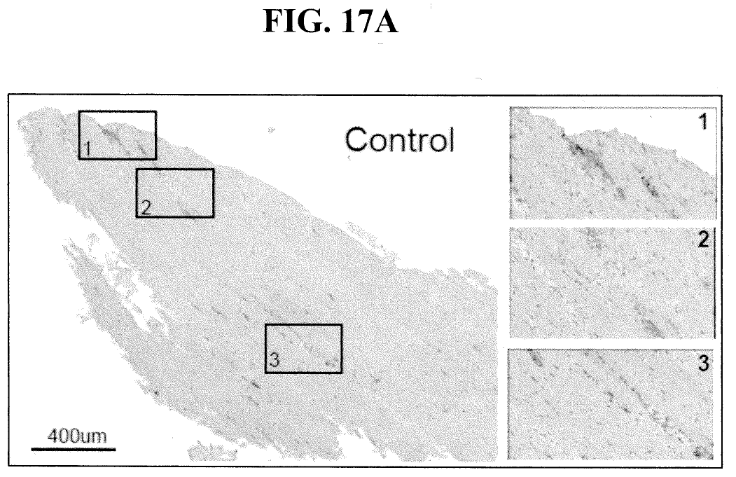

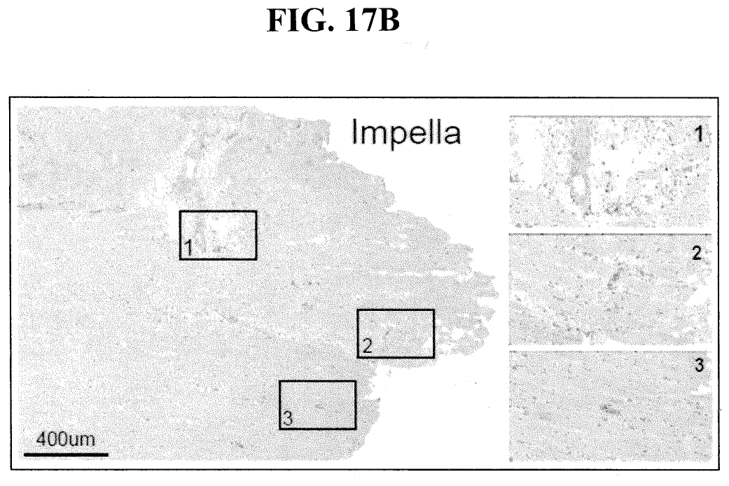

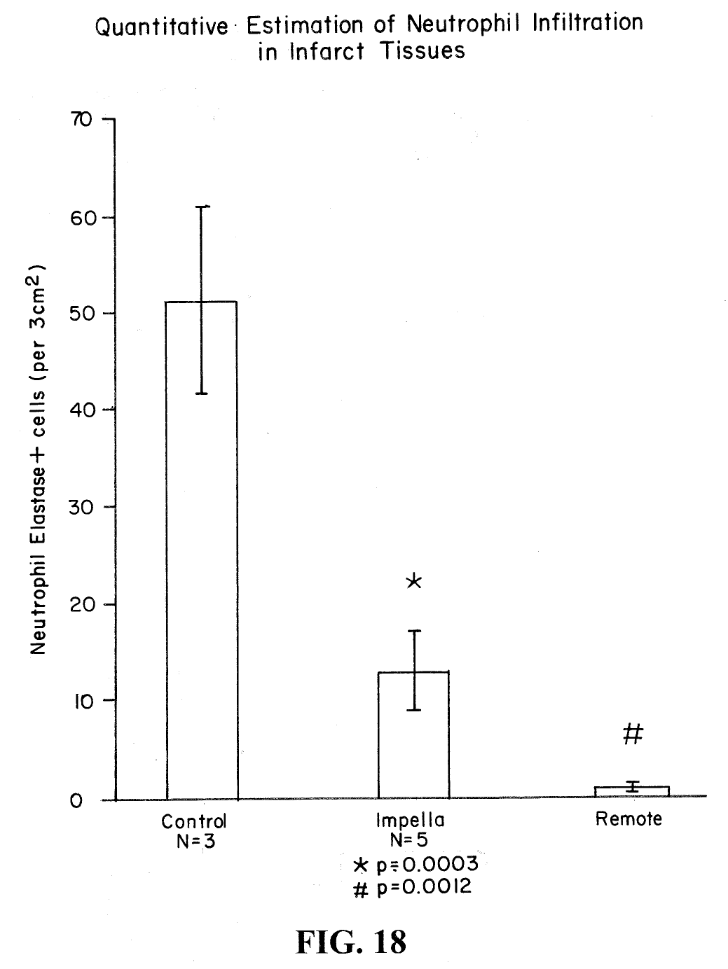

FIG. 17A shows images of tissue of a heart of a swine in the control group exhibiting neutrophil infiltration while FIG. 17B shows images of tissue of a heart of a swine in the Impella group (unloaded) exhibiting reduced neutrophil infiltration. As will be appreciated, a significant reduction of neutrophils reduces the likelihood for MVO due to neutrophil infiltration. The tissue samples of both groups were analyzed and a quantitative estimation of the amount of neutrophils in the infarct tissue was analyzed and plotted. As shown in FIG. 18, the amount of detectable neutrophils in the control group was approximately 52 neutrophil elastate+cells per 3 cm² while the amount of detectable neutrophils in the Impella group was approximately 14 neutrophil elastate+cells per 3 cm² and the amount of detectable neutrophils in the remote tissue was approximately 1 neutrophil elastate+cells per 3 cm². That is, the amount of neutrophils in the Impella group was less than half of that of the control group. Further, the amount of neutrophils in the Impella group was approximately one third of that of the control group.

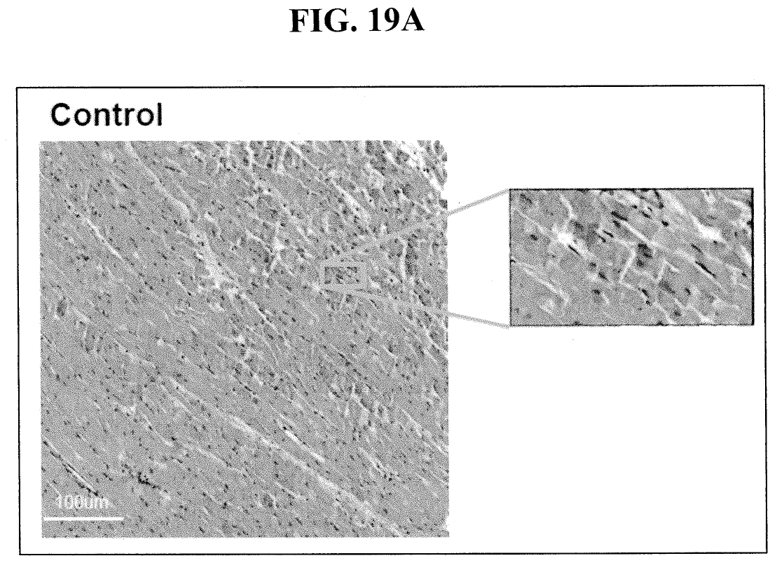

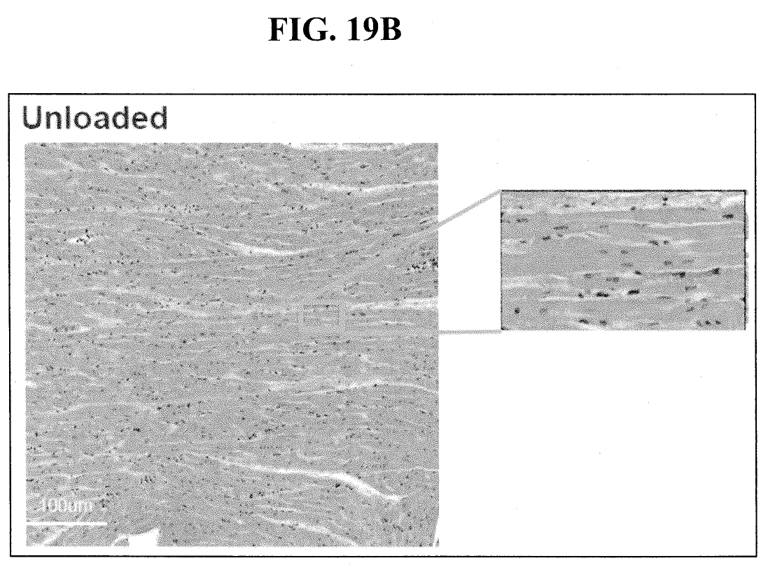

FIG. 19A shows images of tissue of a heart of a swine in the control group exhibiting reduced cardiomyocyte integrity while FIG. 19B shows images of tissue of a heart of a swine in the Impella group (unloaded) exhibiting preserved cardiomyocyte integrity. That is, unloading the heart with an intravalvular pump for a support period t_sp before reperfusion was shown to reduce preserve cardiomyocyte integrity.

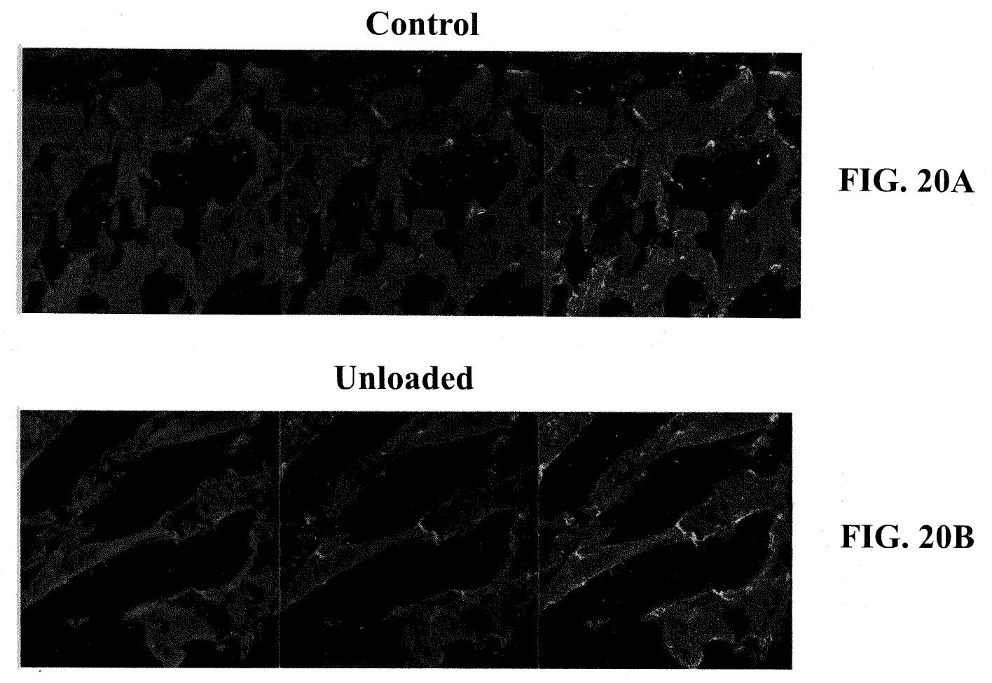

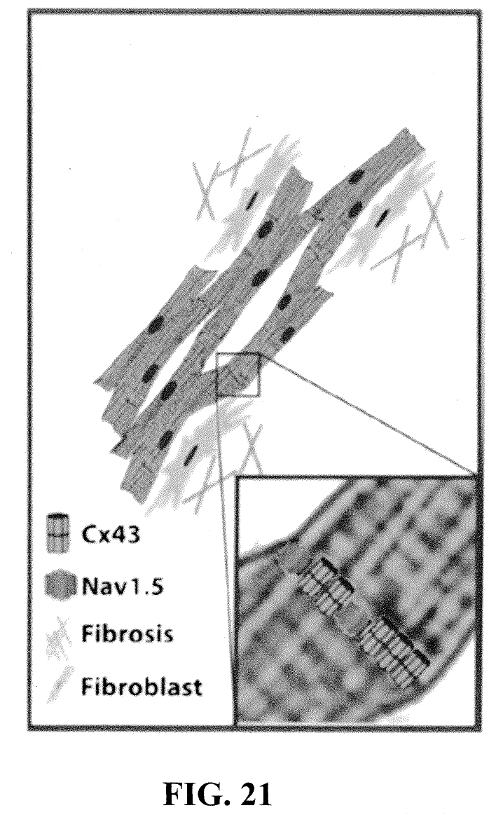

FIG. 20A shows images of tissue of a heart of a swine in the control group exhibiting reduced intercalated disk integrity in cardiomyocytes while FIG. 20B shows images of tissue of a heart of a swine in the Impella group (unloaded) exhibiting preserved intercalated disk integrity in cardiomyocytes. As will be appreciated by one of skill in the art, and as shown further in FIG. 21, intercalated disks comprise Connexin43 and NaV1.5 which can be essential to normal cardiac rhythm. This study shows that unloading the heart for the support period t_sp before reperfusion can help to preserve the Connexin43 and NaV1.5 orientation in the intercalated disks which might offer protection from myocardial infarction induced arrhythmia, both in acute and chronic settings.

One animal from the “No Impella” or control group and one animal from the Impella group (unloaded) were analyzed. There is a 6-fold higher arrythmia burden in the control group as compared to an animal from the Impella group.

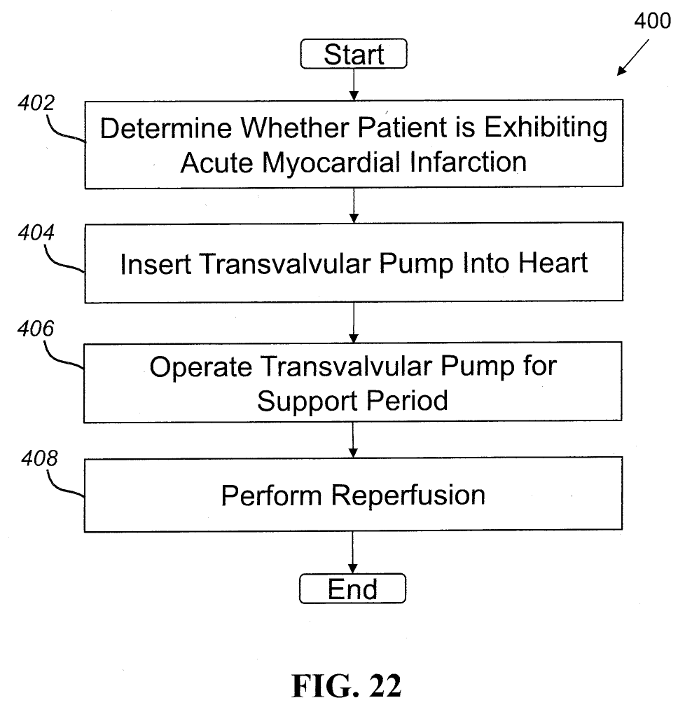

FIG. 22 illustrates a flow chart of a method 400 of treating acute myocardial infarction in a patient, according to an example of the disclosed technology. The method 400 can include determining 402 whether a patient is exhibiting acute myocardial infarction, 404 inserting a transvalvular pump into the heart of the patient and operating 406 the transvalvular pump for at least a support period. Operating 406 the transvalvular pump can be performed according to any of the methods shown and described herein and with the devices shown and described herein. The method 400 can further include performing 408 reperfusion to restore blood flow to the heart. In some examples, the transvalvular pump is operated only for the support period and is ceased prior or simultaneously with reperfusion. In other examples, the transvalvular pump can be operated during the support period, throughout reperfusion, and even following reperfusion for a predetermined time. Further, the method includes reducing the amount of tissue actually comprising microvascular obstruction (MVO) of tissue that is at risk of MVO by approximately 20%.

### Aspects of the Disclosed Technology

The following are exemplary aspects of the disclosure.

Aspect 1: A method of treating acute myocardial infarction in a patient, the method comprising: determining that the patient is exhibiting acute myocardial infarction; inserting a transvalvular pump at least partially into a heart of the patient; operating the transvalvular pump to cause blood to flow at a rate of at least 2.5 L/min for at least a support period; performing a reperfusion of the patient's heart to restore blood flow to the heart after at least the support period has ended; and reducing the amount of tissue actually comprising microvascular obstruction (MVO) of tissue that is at risk of MVO by approximately 20%.

Aspect 2: The method of aspect 1, wherein the support period comprises at least 30 minutes.

Aspect 3: The method of aspect 1, wherein the support period comprises between 30 minutes and 60 minutes.

Aspect 4: The method of aspect 1, wherein the support period comprises greater than 60 minutes.

Aspect 5: The method of any one of aspects 1-4, wherein, of the tissue at risk of MVO, the percentage of the tissue that actually comprises MVO is between 16% and 21%.

Aspect 6: The method of any one of aspects 1-5, wherein the percentage of tissue at risk of MVO is between 40% and 50% of the left ventricle tissue.

Aspect 7: The method of any one of aspects 1-6, further comprising reducing the percentage of tissue comprising infarction due to MVO of the tissue at risk of comprising infarction by approximately 9%.

Aspect 8: The method of aspect 7, wherein the percentage of tissue comprising infarction due to MVO of the tissue at risk of comprising infarction is approximately between 47% and 52 %.

Aspect 9: The method of aspect 8, wherein the percentage of tissue comprising infarction due to MVO of the tissue at risk of comprising infarction is approximately 48%.

Aspect 10: The method of any one of aspects 1-8, further comprising increasing the myocardial salvage index by greater than approximately 9%.

Aspect 11: The method of aspect 10, wherein the myocardial salvage index is approximately between 48% and 53%.

Aspect 12: The method of aspect 11, wherein the myocardial salvage index is approximately 52%.

Aspect 13: The method of any one of aspects 1-12, wherein operating the transvalvular pump results in unloading the heart of the patient.

Aspect 14: The method of any one of aspects 1-13, further comprising preventing noticeable interstitial edema of the tissue of the patient's heart.

Aspect 15: The method of any one of aspects 1-14, further comprising reducing pericapillary edema and diapedesis.

Aspect 16: The method of any one of aspects 1-15, further comprising reducing the amount of detectable neutrophils in the tissue by more than half.

Aspect 17: The method of aspect 16, reducing the amount of detectable neutrophils in the tissue by more than two-thirds.

Aspect 18: The method of any one of aspects 1-17, further comprising preserving cardiomyocyte integrity.

Aspect 19: The method of any one of aspects 1-17, further comprising preserving Connexin43 and NaV1.5 orientation in intercalated disk analysis.

Aspect 20: The method of any one of aspects 1-19, further comprising operating the transvalvular pump for the support period just prior to performing the reperfusion.

Aspect 21: The method of any one of aspects 1-19, operating the transvalvular pump for the support period and during the performing of the reperfusion.

Aspect 22: The method of any one of aspects 1-21, wherein operating the transvalvular pump causes blood to flow at a rate of at least 3.5 L/min for at least the support period.

Aspect 23: A method of treating acute myocardial infarction in a patient, the method comprising: inserting a transvalvular pump at least partially into a heart of the patient; operating the transvalvular pump to cause blood to flow at a rate of at least 2.5 L/min for at least a support period; performing, after the support period, a reperfusion of the patient's heart to restore blood flow to the heart; and reducing a percentage of tissue comprising infarction due to microvascular obstruction (MVO) of tissue at risk of comprising infarction by approximately 9%.

Aspect 24: The method of aspect 23, wherein the support period comprises at least 30 minutes.

Aspect 25: The method of aspect 23, wherein the support period comprises between 30 minutes and 60 minutes.

Aspect 26: The method of aspect 23, wherein the support period comprises greater than 60 minutes.

Aspect 27: The method of any one of aspects 23-26, wherein, of the tissue at risk of MVO, the percentage of the tissue that actually comprises MVO is between 16% and 21%.

Aspect 28: The method of any one of aspects 23-27, wherein the percentage of tissue at risk of MVO is between 40% and 50% of the left ventricle tissue.

Aspect 29: The method of any one of aspects 23-28, further comprising reducing the amount of tissue actually comprising microvascular obstruction (MVO) of tissue that is at risk of MVO by approximately 20%.

Aspect 30: The method of aspect 29, wherein the percentage of tissue comprising infarction due to MVO of the tissue at risk of comprising infarction is approximately between 47% and 52 %.

Aspect 31: The method of aspect 30, wherein the percentage of tissue comprising infarction due to MVO of the tissue at risk of comprising infarction is approximately 48%.

Aspect 32: The method of any one of aspects 23-31, further comprising increasing the myocardial salvage index by greater than approximately 9%.

Aspect 33: The method of aspect 32, wherein the myocardial salvage index is approximately between 48% and 53%.

Aspect 34: The method of aspect 33, wherein the myocardial salvage index is approximately 52%.

Aspect 35: The method of any one of aspects 23-34, wherein operating the transvalvular pump results in unloading the heart of the patient.

Aspect 36: The method of any one of aspects 23-35, further comprising preventing noticeable interstitial edema of the tissue of the patient's heart.

Aspect 37: The method of any one of aspects 23-36, further comprising reducing pericapillary edema and diapedesis.

Aspect 38: The method of any one of aspects 23-37, further comprising reducing the amount of detectable neutrophils in the tissue by more than half.

Aspect 39: The method of aspect 38, reducing the amount of detectable neutrophils in the tissue by more than two-thirds.

Aspect 40: The method of any one of aspects 23-39, further comprising preserving cardiomyocyte integrity.

Aspect 41: The method of any one of aspects 23-39, further comprising preserving Connexin43 and NaV1.5 orientation in intercalated disk analysis.

Aspect 42: The method of any one of aspects 23-41, further comprising operating the transvalvular pump for the support period just prior to performing the reperfusion.

Aspect 43: The method of any one of aspects 23-41, operating the transvalvular pump for the support period and during the performing of the reperfusion.

Aspect 44: The method of any one of aspects 23-43, wherein operating the transvalvular pump causes blood to flow at a rate of at least 3.5 L/min for at least the support period.

Aspect 45: A method of treating acute myocardial infarction in a patient, the method comprising: in response to determining that the patient is exhibiting acute myocardial infarction, inserting a transvalvular pump at least partially into a heart of the patient; operating the transvalvular pump to cause blood to flow at a rate of at least 2.5 L/min for at least a support period; performing a reperfusion of the patient's heart to restore blood flow to the heart; and reducing the amount of detectable neutrophils in tissue of the heart by more than half.

Aspect 46: The method of aspect 45, wherein the support period comprises at least 30 minutes.

Aspect 47: The method of aspect 45, wherein the support period comprises between 30 minutes and 60 minutes.

Aspect 48: The method of aspect 45, wherein the support period comprises greater than 60 minutes.

Aspect 49: The method of any one of aspects 45-48, wherein, of the tissue at risk of MVO, the percentage of the tissue that actually comprises MVO is between 16% and 21%.

Aspect 50: The method of any one of aspects 45-49, wherein the percentage of tissue at risk of MVO is between 40% and 50% of the left ventricle tissue.

Aspect 51: The method of any one of aspects 45-50, further comprising reducing the percentage of tissue comprising infarction due to MVO of the tissue at risk of comprising infarction by approximately 9%.

Aspect 52: The method of aspect 51, wherein the percentage of tissue comprising infarction due to MVO of the tissue at risk of comprising infarction is approximately between 47% and 52 %.

Aspect 53: The method of aspect 52, wherein the percentage of tissue comprising infarction due to MVO of the tissue at risk of comprising infarction is approximately 48%.

Aspect 54: The method of any one of aspects 45-53, further comprising increasing the myocardial salvage index by greater than approximately 9%.

Aspect 55: The method of aspect 54, wherein the myocardial salvage index is approximately between 48% and 53%.

Aspect 56: The method of aspect 45-55, wherein the myocardial salvage index is approximately 52%.

Aspect 57: The method of any one of aspects 45-56, wherein operating the transvalvular pump results in unloading the heart of the patient.

Aspect 58: The method of any one of aspects 45-57, further comprising preventing noticeable interstitial edema of the tissue of the patient's heart.

Aspect 59: The method of any one of aspects 45-58, further comprising reducing pericapillary edema and diapedesis.

Aspect 60: The method of any one of aspects 45-59, further comprising reducing the amount of tissue actually comprising microvascular obstruction (MVO) of tissue that is at risk of MVO by approximately 20%.

Aspect 61: The method of aspect 60, reducing the amount of detectable neutrophils in the tissue by more than two-thirds.

Aspect 62: The method of any one of aspects 45-61, further comprising preserving cardiomyocyte integrity.

Aspect 63: The method of any one of aspects 45-62, further comprising preserving Connexin43 and NaV1.5 orientation in intercalated disk analysis.

Aspect 64: The method of any one of aspects 45-63, further comprising operating the transvalvular pump for the support period just prior to performing the reperfusion.

Aspect 65: The method of any one of aspects 45-63, operating the transvalvular pump for the support period and during the performing of the reperfusion.

Aspect 66: The method of any one of aspects 45-65, wherein operating the transvalvular pump causes blood to flow at a rate of at least 3.5 L/min for at least the support period.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. A method of treating acute myocardial infarction in a patient, the method comprising:
determining that the patient is exhibiting acute myocardial infarction; inserting a transvalvular pump at least partially into a heart of the patient;
operating the transvalvular pump to cause blood to flow at a rate of at least 2.5 L/min for at least a support period;
performing a reperfusion of the patient's heart to restore blood flow to the heart after at least the support period has ended; and
reducing the amount of tissue actually comprising microvascular obstruction (MVO) of tissue that is at risk of MVO by approximately 20%.

2. The method of claim 1, wherein, of the tissue at risk of MVO, the percentage of the tissue that actually comprises MVO is between 16% and 21%.

3. The method of claim 2, wherein the percentage of tissue at risk of MVO is between 40% and 50% of the left ventricle tissue.

4. The method of claim 1, further comprising reducing the percentage of tissue comprising infarction due to MVO of the tissue at risk of comprising infarction by approximately 9%.

5. The method of claim 4, wherein the percentage of tissue comprising infarction due to MVO of the tissue at risk of comprising infarction is approximately between 47% and 52%.

6. The method of claim 5, wherein the percentage of tissue comprising infarction due to MVO of the tissue at risk of comprising infarction is approximately 48%.

7. The method of claim 1, further comprising increasing the myocardial salvage index by greater than approximately 9%.

8. The method of claim 7, wherein the myocardial salvage index is approximately between 48% and 53%.

9. The method of claim 8, wherein the myocardial salvage index is approximately 52%.

10. A method of treating acute myocardial infarction in a patient, the method comprising:
inserting a transvalvular pump at least partially into a heart of the patient;
operating the transvalvular pump to cause blood to flow at a rate of at least 2.5 L/min for at least a support period;
performing, after the support period, a reperfusion of the patient's heart to restore blood flow to the heart; and
reducing a percentage of tissue comprising infarction due to microvascular obstruction (MVO) of tissue at risk of comprising infarction by approximately 9%.

11. The method of claim 10, wherein, of the tissue at risk of MVO, the percentage of the tissue that actually comprises MVO is between 16% and 21%.

12. The method of claim 11, wherein the percentage of tissue at risk of MVO is between 40% and 50% of the left ventricle tissue.

13. The method of claim 1, further comprising reducing the amount of tissue actually comprising microvascular obstruction (MVO) of tissue that is at risk of MVO by approximately 20%.

14. The method of claim 13, wherein the percentage of tissue comprising infarction due to MVO of the tissue at risk of comprising infarction is approximately between 47% and 52 %.

15. The method of claim 14, wherein the percentage of tissue comprising infarction due to MVO of the tissue at risk of comprising infarction is approximately 48%.

16. The method of any claim 1, further comprising increasing the myocardial salvage index by greater than approximately 9%.

17. The method of claim 16, wherein the myocardial salvage index is approximately between 48% and 53%.

18. The method of claim 17, wherein the myocardial salvage index is approximately 52%.

19. A method of treating acute myocardial infarction in a patient, the method comprising:
in response to determining that the patient is exhibiting acute myocardial infarction, inserting a transvalvular pump at least partially into a heart of the patient;
operating the transvalvular pump to cause blood to flow at a rate of at least 2.5 L/min for at least a support period;
performing a reperfusion of the patient's heart to restore blood flow to the heart; and
reducing the amount of detectable neutrophils in tissue of the heart by more than half.

20. The method of claim 19, wherein, of the tissue at risk of MVO, the percentage of the tissue that actually comprises MVO is between 16% and 21%.
